(19) United States
(12) Patent Application Publication
(10) Pub. No.: US 2021/0407695 A1
(43) Pub. Date: Dec. 30, 2021

(54) METHOD, APPARATUS, AND COMPUTER-READABLE MEDIUM FOR PROVIDING MOTIVATION TO SEEK AND GET TREATMENT

(71) Applicant: Chess Mobile Health, Inc., East Rochester, NY (US)

(72) Inventor: Hans Morefield, Katonah, NY (US)

(21) Appl. No.: 16/910,205

(22) Filed: Jun. 24, 2020

(51) Int. Cl. G16H 80/00; G16H 50/30; G16H 20/00; H04L 12/58

(52) U.S. Cl. CPC G16H 80/00; H04L 51/10; G16H 20/00; G16H 50/30

(57) ABSTRACT

Embodiments of the present disclosure provide a method, apparatus and computer-readable medium of providing motivation to get treatment. An exemplary method includes receiving, by a processor, patient referral data of a patient from a referring entity, and transmitting, to a user equipment (UE), an invitation, wherein the UE is associated with the patient. An exemplary method further includes receiving, by the processor, a login of the patient in response to the invitation, and in response to the receiving, determining, by the processor, a subset of a plurality of videos from a plurality of sources to send to the UE based on a correspondence between the patient referral data and the subset of the plurality of videos. An exemplary method still further includes displaying, by the processor, the subset of videos of the plurality of videos, wherein the subset of videos can be prioritized and re-prioritized based on feedback including actions of the patient as well as others.

### BACKGROUND OF THE INVENTION

### Field of the Invention

The present invention relates generally to a method, apparatus and computer-readable medium for providing motivation to seek and to get treatment.

### Description of Related Art

Addiction is a treatable and chronic medical disease. The disease is associated with complex interactions between an individual's brain, their genetics, their environment and life experiences. Individuals suffering from addiction use substances or engage in behaviors that become compulsive and often continue despite harmful consequences to the individual, their families, friends, and their community. Some of the types of drug and behavioral addiction include amphetamine addiction, alcoholism, opioid addiction, food addiction, addiction to narcotics, video game addiction, gambling addiction, and sexual addiction.

There are a number of risk factors that can increase an individual's risk for developing an addiction. Some of these risk factors include the genetic makeup of the individual. The genetic makeup of an individual can make an individual more or less prone to develop addictions. Environmental factors, such as the experiences of an individual can contribute to the likelihood that an individual will develop addictions. The age of the individual along with mental health disorders, and epigenetic factors can also add to the likelihood of whether an individual is predisposed to addictive behaviors.

Treatment of addiction can take many forms. Some treatments involve pharmacological or biological based treatments. Other types of addiction treatment include addiction rehabilitation, individual and group psychotherapy, behavior-modification strategies, twelve-step programs and residential treatment facilities. Despite the vast number of treatments available, the disease of addiction persists.

### BRIEF SUMMARY OF THE INVENTION

In view of the foregoing, it is an object of the present disclosure to provide a method, apparatus and computer-readable medium of providing motivation to an individual to seek and get treatment.

A first exemplary embodiment of the present disclosure provides a method of providing motivation to get treatment, the method includes receiving, by a processor, patient referral data of a patient from a referring entity, and transmitting, to a user equipment (UE), an invitation, wherein the UE is associated with the patient. The method further includes receiving, by the processor, a login of the patient in response to the invitation. The method still further includes in response to the receiving, determining, by the processor, a subset of a plurality of videos from a plurality of sources to send to the UE based on a correspondence between the patient referral data and the subset of the plurality of videos, and displaying, by the processor, the subset of videos of the plurality of videos.

A second exemplary embodiments of the present disclosure provides an apparatus including at least one processor and at least one memory storing computer program instructions executable by the at least one processor, wherein the at least one memory and the computer program instructions and the at least one processor are configured to cause the apparatus to at least receive patient referral data of a patient from a referring entity. The at least one memory and the computer program instructions and the at least one processor are configured to cause the apparatus to at least transmit, to a user equipment (UE), an invitation, wherein the UE is associated with the patient, and receive a login of the patient in response to the invitation. The at least one memory and the computer program instructions and the at least one processor are configured to cause the apparatus to at least in response to the receiving, determine a subset of a plurality of videos from a plurality of sources to send to the UE based on a correspondence between the patient referral data and the subset of the plurality of videos.

A third exemplary embodiment of the present disclosure provides a non-transitory computer-readable medium tangibly storing computer program instructions which when executed by a processor, cause the processor to at least receive patient referral data of a patient from a referring entity, and transmit, to a user equipment (UE), an invitation, wherein the UE is associated with the patient. The computer program instructions which when executed by a processor, further cause the processor to at least receive a login of the patient in response to the invitation, and in response to the receiving, determine a subset of a plurality of videos from a plurality of sources to send to the UE based on a correspondence between the patient referral data and the subset of the plurality of videos.

A fourth exemplary embodiment of the present disclosure provides a method including receiving, by the processor, a login of the patient from a user equipment (UE), wherein the patient is associated with patient referral data, determining, by the processor, a subset of a plurality of videos from a plurality of sources to send to the UE based on a correspondence between the patient referral data and the subset of the plurality of videos, and displaying, by the processor, the subset of videos of the plurality of videos.

A fifth exemplary embodiment of the present disclosure provides an apparatus including at least one processor and at least one memory storing computer program instructions executable by the at least one processor, wherein the at least one memory and the computer program instructions and the at least one processor are configured to cause the apparatus to at least receive a login of the patient, determine a subset of a plurality of videos from a plurality of sources to send to the UE based on a correspondence between the patient referral data and the subset of the plurality of videos, and displaying, by the processor, the subset of videos of the plurality of videos.

A sixth exemplary embodiment of the present disclosure provides a non-transitory computer-readable medium tangibly storing computer program instructions which when executed by a processor, cause the processor to at least receive a login of the patient, determine a subset of a plurality of videos from a plurality of sources to send to the UE based on a correspondence between the patient referral data and the subset of the plurality of videos, and displaying, by the processor, the subset of videos of the plurality of videos.

The following will describe embodiments of the present disclosure, but it should be appreciated that the present disclosure is not limited to the described embodiments and various modifications of the invention are possible without departing from the basic principles. The scope of the present disclosure is therefore to be determined solely by the appended claims.

### BRIEF DESCRIPTION OF THE SEVERAL VIEWS OF THE DRAWING(S)

FIG. 1 presents an exemplary signaling diagram suitable for use in practicing exemplary embodiments of this disclosure.

FIG. 2A presents a logic flow diagram suitable for use in practicing exemplary embodiments of this disclosure.

FIG. 2B presents another logic flow diagram suitable for use in practicing exemplary embodiments of this disclosure.

FIG. 3 presents an exemplary block diagram suitable for use in practicing exemplary embodiments of this disclosure.

FIG. 4 presents a logic flow diagram in accordance with a method, apparatus, and computer-readable medium for performing exemplary embodiments of this disclosure.

FIG. 5 presents another logic flow diagram in accordance with an alternative embodiment of a method, apparatus, and computer-readable medium for performing exemplary embodiments of this disclosure.

### DETAILED DESCRIPTION OF THE INVENTION

Addiction as well as other diseases require treatment by multiple medical professionals. This can often require the patient to meet with medical professionals at a number of different locations. With each change of location or each time that a patient needs to go to a different location to meet with a medical professional there is a chance or likelihood that the patient will not attend the meeting with medical professional. For instance, a patient seeking treatment for an addiction may obtain a referral from a doctor, nurse, or other medical professional at a hospital, from their primary care physician, or other referring medical professional to attend an addiction treatment facility. Embodiments of an addiction treatment facility include residential treatment facilities. In this example, in order for the patient to be treated as recommended by the referring medical professional, the patient would need to leave the location in which they received the referral and travel to the location of the addiction treatment entity. The time it takes the patient to decide (i) to attend the treatment entity, (ii) to travel to the location of the addiction treatment entity, (iii) to pack the appropriate materials for their stay at the treatment entity, (iv) whether they think they need treatment, (v) whether they think they will benefit from treatment, and/or (vi) to make appropriate arrangements with work, family, friends, and/or acquaintances such that the patient's affairs are in order for him/her to be away is more time for the patient to decide to stop the referred treatment, stop seeking the referred treatment, not attend the treatment entity or worse an opportunity for the patient to relapse or continue to feed their addiction. Embodiments of the present disclosure seek to increase the likelihood that the patient will seek and get the treatment to which they are referred. Embodiments of the present disclosure are applicable to patients seeking treatment for addiction, over eating, anorexia, bulimia, binge eating, dieting disorders, and patients who have been referred to receive additional medical services (e.g., surgeries, diagnostics, tests, etc.)

Embodiments of the present disclosure provide a method, apparatus and computer-readable medium for increasing the likelihood that patients attend prescribed addiction treatment. Embodiments of the present disclosure provide a method, apparatus, and computer-readable medium for referral management. Embodiments provide a process that increases the likelihood that patients obtain the treatment to which they are referred by providing determined video and/or audio content to the patient (via an electronic device) from a plurality of sources in response to a referral. Embodiments provide a method and apparatus for providing a subset of video content from a plurality of videos from a plurality of sources to a patient that has been referred to a treatment entity by a referring entity based on (i) patient referral data and (ii) the subject matter of the plurality of videos. Embodiments of the plurality of videos include educational, motivational, and/or testimonial videos. Embodiments of educational videos include videos that provide instructional, teaching or educational information regarding a particular topic including addiction generally, consequences of addiction, addiction treatment, addiction recovery, information regarding one or more particular detox centers, and/or one or more particular treatment centers. Embodiments of motivational videos include information meant to encourage individuals to perform a particular action including getting treatment for addiction or other medical issue. Embodiments of testimonial videos include information from individuals regarding a particular action they have taken including their attendance to a given treatment entity such as but not limited to an addiction treatment center, detox center or other medical facility. Embodiments provide a method and apparatus for providing a subset of videos from a plurality of videos to a patient based on a correspondence between patient referral data and the content of the subset of videos. Embodiments of the present disclosure provide for receiving feedback data from a patient's user equipment (UE) that is associated with a viewing history of the subset of videos and the ability to update the correspondence between the subset of videos relative to patient referral data. Embodiments of the present disclosure provide a method and apparatus for enrolling a patient into an application on a UE by sending an invitation to the patient in response to receiving a referral of the patient to a treatment entity. Embodiments of the present disclosure provide a method and apparatus for providing video content to patients from a plurality of different sources located in different locations.

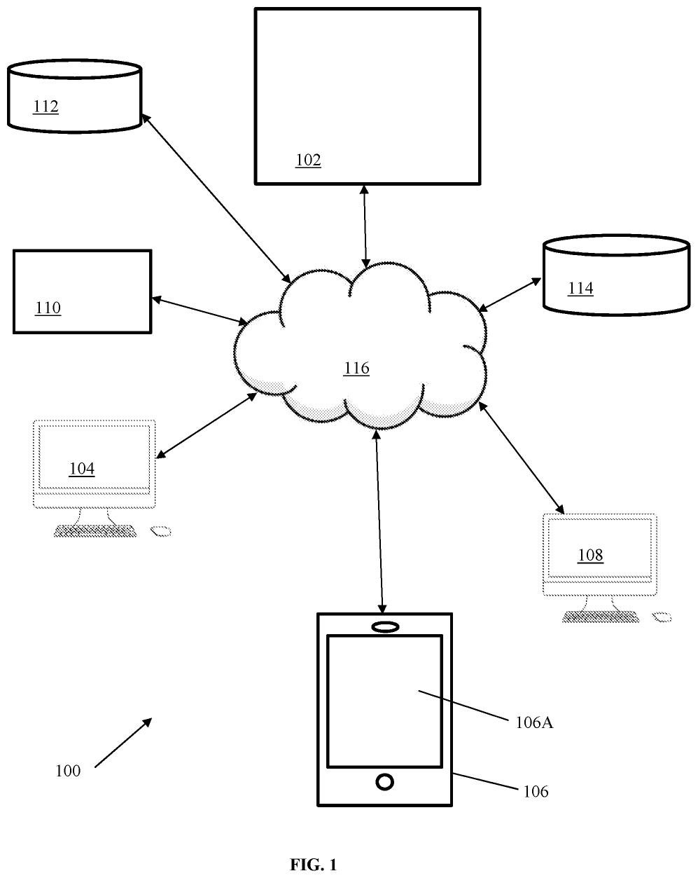

Referring to FIG. 1, shown is an exemplary signaling diagram between exemplary devices suitable for use in practicing exemplary embodiments of this disclosure. Shown in FIG. 1 is referral system 100. Referral system 100 includes server 102, referring entity 104, patient user equipment (UE) 106, treatment entity 108, video storage server 110, third party video sources 112 and 114, and network 116. Embodiments of server 102 include a processor and a memory storing computer program instructions, which when executed by the processor of server 102 cause the server to perform as described herein. Embodiments of server 102 can include one or multiple servers. Server 102 is operable for (wired or wireless) bidirectional communication via network 116 with referring entity 104, patient user equipment (UE) 106, treatment entity 108, video storage server 110, and third party video sources 112 and 114. Embodiments of network 116 include the internet, cellular networks, WIFI networks, public and private networks, wide area networks, and local area networks.

Embodiments of referring entity 104 include hospitals, primary care physicians, addiction treatment facilities, urgent care facilities, or any other type of medical facility that is able to refer patients for further medical treatment, such as for the treatment of drug and alcohol addiction. Embodiments of referring entity 104 have one or more computers, laptops, smartphones, tablets, wearable electronic devices, or other electronic devices that allow the referring entity 104 to conduct (wired or wireless) bidirectional communication with server 102, UE 106, and treatment entity 108 (via network 116) to send and receive data. Embodiments of data include patient referral data, which can include the name of the patient, the date of birth of the patient, medical history of the patient, a diagnosis of the patient, the medical services provided to the patient, the medical services that the patient has been referred to receive, the gender of the patient, the age of the patient, the geographic location of the patient, the number of times the patient has relapsed (for the case that the patient is being treated for addiction or similar disease), allergies of the patient, the family medical history of the patient, or any other relevant information regarding the patient. Embodiments of referring entity 104 can include or maintain one or more video and/or audio files that can be transmitted to UE 106, video storage server 110 and/or server 102.

Embodiments of patient UE 106 includes any type of electronic device that is operable by a patient to send and receive electronic data from server 102 and is operable to play, display or have displayed video and/or audio files or content. Exemplary embodiments of UE 106 include mobile phones, smartphones, electronic wearable devices, virtual or augmented reality devices, tablets, and computers. Embodiments of UE 106 include a processor, a memory able to store computer program instructions, a user interface, a display and is operable for (wired or wireless) bidirectional communication such that it can send and receive data from server 102, referring entity 104, and treatment entity 108 (via network 116). Embodiments of bidirectional communication include sending and receiving emails, text messages, video messages, and/or other electronic messaging services. Embodiments of UE 106 include a user interface/display 106A that allow a user to operate UE 106 and to display and/or play video and/or audio content, clips, files and the like.

Embodiments of treatment entity 108 include addiction treatment centers, recovery facilities, hospitals, urgent care facilities, medical facilities, and the like. Embodiments of treatment entity 108 have one or more computers, laptops, smartphones, tablets, wearable electronic devices, or other electronic devices that allow the treatment entity 108 to conduct (wired or wireless) bidirectional communication with server 102 (via network 116) to send and receive data. Embodiments of the data that the treatment entity 108 is operable to send and receive include patient referral data and data regarding the treatment of the patient. Embodiments of data regarding the treatment of the patient includes data corresponding to enrollment of the patient at the treatment facility and/or treatment of the patient at the treatment entity 108. Embodiments of treatment entity 108 can include one or more video and/or audio files that can be transmitted to UE 106, video storage server 110 and/or server 102.

Embodiments of video storage server 110 include a processor and a memory storing computer program instructions, which when executed by the processor of server 110 cause the video storage server 110 to operate as set forth herein. Video storage server 110 is operable for (wired or wireless) bidirectional communication with server 102 and UE 106 via network 116 to send and receive data. Embodiments of video storage server 110 are operable to maintain or store a plurality of video and/or audio files such that that plurality of video and/or audio files are associated with a plurality of categories. Embodiments of the plurality of categories include the subject matter (e.g., type of addiction) referenced in the video and/or audio content or files, length of the video and/or audio file, the age of the recommended viewer associated with the file, and/or the feedback (e.g., positive or negative feedback received from prior patients) associated with the video and/or audio file. It should be appreciated that embodiments include video storage server 110 being operable to recategorize the plurality of video and/or audio files. In other words, embodiments include updating the plurality of categories associated with each one of the plurality of videos based on changes to the video and/or audio files, feedback received from users who have viewed or listened to the video and/or audio files, and whether or not the videos and/or audio files have been or not been associated with users that successfully obtained the treatment to which they were referred. Embodiments of video storage server 110 can include one or more servers. Embodiments of video storage server 110 are operable to transmit one or more video and/or audio files to UE 106 (via network 116) for viewing (and/or listening) by the patient associated with UE 106 in response to instructions from server 102. It should be appreciated that while video storage server 110 is a separate device from server 102, embodiments include video storage server 110 being an element or part of server 102.

Embodiments of third party video sources 112 and 114 include third party resources that maintain publicly accessible and privately accessible video and/or audio files or content. Exemplary embodiments of third party video sources 112 and 114 include websites (e.g., YouTube, Google, Vimeo, Twitch, Vube, DailyMotion, etc.), online libraries, online medical websites, research institutions, and the like. Embodiments of third party video sources 112 and 114 provide video and/or audio files that can be accessed by server 102 such that the video and/or audio files can be categorized based on their content, subject matter, feedback, and/or typical viewing audience. Embodiments of third party video sources 112 and 114 provide video and/or audio files that can be accessed by server 102 such that the video and/or audio files can be transmitted to UE 106. Embodiments of third party video sources 112 and 114 are operable for bidirectional communication with server 102 and/or UE 106 via network 116. Embodiments of third party video sources 112 and 114 can include one or more servers having one or more processors and memories.

In practice, the referring entity 104 will refer the patient associated with UE 106 to a treatment entity 108. It should be appreciated that in some embodiments the referring entity 104 and the treatment entity 108 are the same entity. In other words, embodiments include the referring entity 104 referring the patient associated with UE 106 to itself the referring entity 104. The referring entity 104 will transmit patient referral data to server 102. Server 102 will then transmit to UE 106 an invitation. Embodiments of an invitation include an email with a (i) link to download an application, and/or (ii) a link to a website, a text message with a (i) link to download an application, and/or (ii) a link to a website, or an electronic message with a (i) link to download an application, and/or (ii) a link to a website. In some embodiments, the referring entity 104 and/or server 102 may transmit a message to the treatment entity 108 in response to receiving the patient referral data in order to put the treatment entity 108 on notice of the referral and receipt of the patient referral information. The user of UE 106 will then click on the link or accept the invitation by downloading the application or going to the website. The user of UE 106 will then be prompted to login to the application or website with identifying information. Embodiments of identifying information can include a username, password, date of birth, social security number or unique identifying number provided to the user in the invitation. Server 102 will receive a notification from the application or website that the user has logged into the application or website.

In response to receiving the login or notification of the login by the user, server 102 will determine whether any of the video and/or audio files from the video storage server 110, referral entity 104, treatment entity 108 and/or third party video sources 112, 114 have a correspondence with the patient referral data. Embodiments of a correspondence can be based on at least one of the particular treatment entity 108 (e.g., does the treatment entity mainly treat alcohol addiction, drug addiction, depression, age of the typical patient, location of the treatment entity, etc.), the diagnosis of the patient, the patient medical history, the types of services that will be provided to the patient by treatment entity 108, the gender of the patient, the age of the patient, the geographic location of the patient and the treatment entity 108, the prior treatment history of the patient, the content and/or category of the video and/or audio files, and feedback from prior patients to the video and/or audio files. In other words, server 102 will assess the video and/or audio files that are available on video storage server 110, referral entity 104, treatment entity 108 and/or third party video sources 112, 114 and will determine whether that content is suitable for encouraging the patient to attend the treatment entity 108. In one embodiment, each of the video and/or audio files will include information (e.g., title information, metadata, short description, feedback data, prior patient comments, age of individuals in video, type of addiction/treatment in video, the particular treatment entity in video, etc.) that is associated with the file. Server 102 will determine the content of the video and/or audio files based on the included information, and will use that included information to determine whether there is a correspondence with the patient referral data. For instance, server 102 might have access to video A and video B. Video A might concern an individual seeking recovery services from alcohol abuse for women between the ages of 20-30 years of age in the downtown Rochester, N.Y. Video B might concern an individual seeking addiction recovery services for opioids for men between the ages of 60-70 years of age in the suburbs of Buffalo, N.Y. If the patient in this instance was a 25-year-old women located in Rochester, N.Y. suffering from alcoholism, server 102 may determine to transmit video A to the patient rather than video B. In this example, it should be appreciated that video A and video B may be located in different places from different sources.

In another example, server 102 may have access to video C and video D. Video C may concern an individual seeking treatment for narcotics for men between the ages of 40-50 years. Video C may have received positive feedback from prior patients (e.g., 4 out of 5 stars), however, 75% of the patients who previously viewed video C did not attend the treatment entity 108 to which they were referred. Video D may also concern an individual seeking treatment for narcotics for men between the ages of 40-50 years. Video D may have received feedback from prior patients of 1 out of 5 stars, but 80% of patients who previously viewed video D actually attended the treatment entity 108 to which they were referred. In this example, server 102 may determine to transmit video D over video C even though prior patient feedback was so positive. It should be appreciated that the above examples are purely exemplary embodiments of the determining by server 102. As such, it should be appreciated that the determining of which videos and/or audio files to transmit to UE 106 by server 102 can be based on a number of factors as set forth herein, and no one factor provides a bright line test for determining which video and/or audio files to transmit to UE 106 and which video and/or audio files to not transmit to UE 106.

In another embodiment, server 102 will analyze each of the video and/or audio files to determine the content of the video and/or audio files in order to determine the subject matter (e.g., the type of addiction addressed in the video/audio) of the video and/or audio files. In yet another embodiment, server 102 will determine the content of the video and/or audio files prior receiving the login from the patient. In this embodiment, server 102 will associate each of the video and/or audio files with their subject matter, category or content. Once the server 102 has determined whether there are any video and/or audio files that have a correspondence to the patient referral data, server 102 will transmit the video and/or audio files to UE 106. In one embodiment, once the server 102 has determined whether there are any videos and/or audio files that have a correspondence to the patient referral data, in some embodiments, server 102 will prioritize the order in which the selected video and/or audio files are presented, displayed or sent to UE 106 based on a strength of the correspondence. In other words, embodiments include server 102 associating a ranking or score with each of the selected videos and/or audio files based on the strength of the correspondence. For instance, server 102 may determine that Videos A, B, C, and D all have a correspondence to the patient referral data. However, server 102 may determine that based on the strength of the correspondence or scoring, the videos should be prioritized in the following order B, D, A, C. In this instance, Video B would have a higher strength or score than D, A, and C, Video D would have a higher strength or score than A and C, and Video A would have a higher strength or score than Video C. The server 102 can then present the videos to the UE 106 in the order B, D, A, C. UE 106 will then display one or more of the received video and/or audio files. In one embodiment, UE 106 may automatically display the received video and/or audio files or may display them in response to a user input.

UE 106 will be operable to transmit user feedback data to server 102 based on the actions of the patient on UE 106. For instance, UE 106 may transmit feedback data associated with viewing history of the video and/or audio files, the particular video and/or audio files the patient viewed, whether the patient viewed the entire video and/or audio file, the particular video and/or audio file that the patient did not view, any comments the patient entered into the UE 106 after viewing a particular video and/or audio file, any type of rating that the patient gave to the video and/or audio files viewed (e.g., 1 out of 5 stars, 2 out of 5 stars, 4 out of 5 stars, etc.), and whether the patient in fact attended the treatment entity 108 after viewing the video and/or audio file. Server 102 will then update the information associated with the viewed video and/or audio file based on the received feedback such that server 102 can take into account the feedback information next time it receives patient referral data.

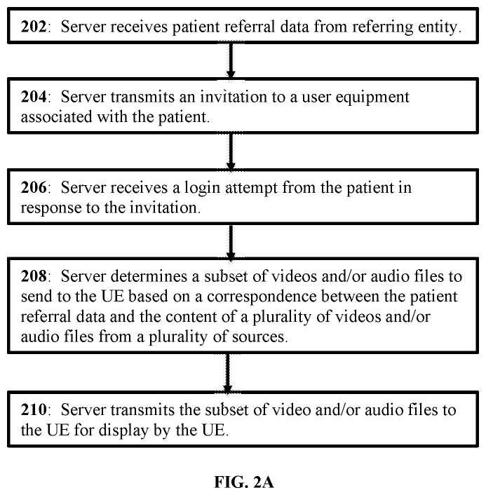

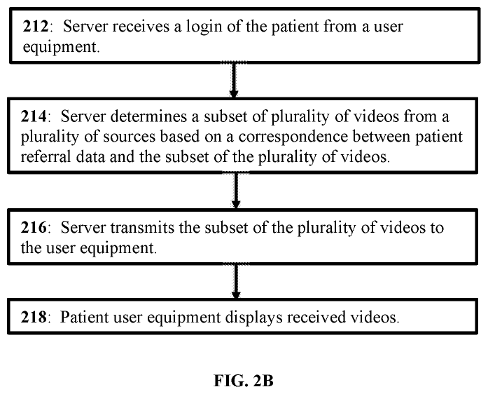

Reference is now made to FIG. 2A, which illustrates an exemplary simplified logic flow diagram in accordance with an exemplary process or method of this disclosure. The process begins at block 202 which states that the server receives patient referral data from the referring entity. In response to receiving the patient referral data the server will then at block 204 transmit an invitation to a user equipment associated with the patient. The patient on the user equipment will then respond to the invitation by clicking a link, downloading an application and logging in with their credentials. When the patient attempts to login, the server at block 206 will receive a login attempt from the patient in response to the invitation. Next, at block 208, the server will determine which subset of videos and/or audio files to send to the user equipment of the patient based on whether there is a correspondence between the patient referral data and the content of a plurality of videos and/or audio files, wherein the plurality of video and/or audio files are from a plurality of different sources. Finally, at block 210 the server will transmit the subset of video and/or audio files to the user equipment such that they can be displayed, viewed or played on the user equipment by the patient.

It should be appreciated that the plurality of different sources may be in the same geographic location and may be in different geographic locations. It should also be appreciated that embodiments include the determining step (i.e., block 208) occurring in response to receiving patient referral data from the referring entity (block 202) rather than in response to the server receiving a login attempt from the patient in response to the invitation (block 206). Embodiments of a login attempt include the patient's user equipment logging into a website linked to the invitation or downloading an application and logging into the application. In this embodiment, the server will determine the subset of video and/or audio files that have a correspondence to the patient referral data in response to receiving the patient referral data. Contemporaneous with the determining step, the server will transmit the invitation to the user equipment of the patient. When the server receives a login attempt from the patient in response to the invitation, the server will simply transmit the determined subset of video and/or audio files to the user equipment of the patient such that they can be played by the user equipment. In this regard, the transmission of video and/or audio files to the user equipment of the patient can be nearly instantaneous in response to the user's login to the website, application and the like.

Referring to FIG. 2B, shown is another logic flow diagram in accordance with embodiments of this disclosure. Shown in FIG. 2B is an exemplary process in which the patient has already previously logged into the website or application in response to an invitation, but has now returned to the website or application after the initial login. The exemplary process begins at block 212 which states that the server will receive a login of the patient from their user equipment. It should be appreciated that the login attempt can be from the same or a different user equipment provided that the patient enters the same login credentials as they did originally. Next at block 214, the server determines a subset of plurality of videos from a plurality of sources based on a correspondence between the patient referral data and the subset of the plurality of videos. It should be appreciated that embodiments include the server determining the subset of plurality of videos each time the patient logs into the website or application. In another embodiment the server will store the subset of plurality of videos from the original login attempt of the patient such that each time the patient logs into the website or application the server can retrieve this information rather than determine it during each login. In another embodiment, the server will alter the subset of plurality of videos each time the patient logs into the website or application such that the subset of the plurality of videos are (i) prioritized in a different order than that previously presented to the patient's user equipment, (ii) exclusively new videos that were not previously presented the patient's user equipment, or (iii) previously presented videos and new videos that were not previously presented to the patient's user equipment. In this embodiment, the server is operable to determine whether to transmit the same videos, new videos or a combination of new and previously sent videos based on (i) the patient referral data, (ii) what videos were previously viewed by the user of the patient user equipment, (iii) a new referral received by the patient for new or additional treatment, and/or (iv) feedback received on the videos. It should also be appreciated the server is operable to maintain or store the determined subset of plurality of videos until the patient receives treatment at the treatment entity, is released from the treatment entity, or receives a new patient referral. At block 216, the server transmits the subset of the plurality of videos to the patient's user equipment. Then at block 218, the patient user equipment displays the received videos.

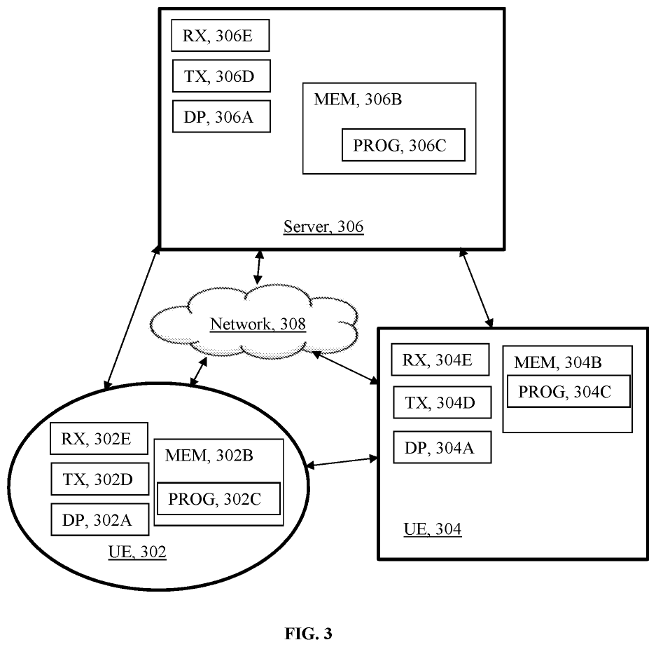

Referring to FIG. 3, shown is a simplified block diagram of the various electronic devices that are suitable for use in practicing exemplary embodiments of the present disclosure. In FIG. 3 is server 306 adapted for bidirectional communication with user equipment (UE) 302 either indirectly through network 308 or directly through wired or wireless means (e.g., Bluetooth). Server 306 is also adapted for bidirectional communication with user equipment (UE) 304 either indirectly through network 308 or directly through wired or wireless means. It should be appreciated that embodiments of server 306 include both a single server or a plurality of server.

UE 302 may include processing means such as a processing system and/or at least one data processor (DP) 306A, storing means such as at least one computer-readable medium or computer-readable memory (MEM) 302B storing at least one computer program (PROG) 302C, and also communicating means such as a transmitter (TX) 302D and receiver (RX) 302E for bidirectional wireless communication with server 306 and/or UE 304 and/or any other UE's or devices (not shown). It should be appreciated that embodiments of UE 302 include a patient's UE.

Server 306 includes its own processing means such as a processing system and/or at least one data processor (DP) 306A, storing means such as at least one computer-readable memory (MEM) 306B storing at least one computer program (PROG) 306C, and communication means such as a transmitter (TX) 306D and a receiver (RX) 306E for bidirectional wireless communications with other devices as known in the art.

Similarly, UE 304 includes its own processing means such as processing system and/or at least one data processor (DP) 304A, storing means such as at least one computer-readable memory (MEM) 304B storing at least one computer program (PROG) 304C, and communication means such as a transmitter (TX) 304D and a receiver (RX) 304E for bidirectional wireless communications with other devices. It should be appreciated that embodiments of UE 304 can include any type of UE operable by a referring entity and/or a treatment entity.

Various embodiments of UE 302 can include, but are not limited to: cellular telephones, mobile phones, smartphones, laptop and desktop computers, personal portable digital devices having wireless communication capabilities including, but not limited to wearable devices and smart watches.

At least one of the PROGs 302C, 304C, or 306C includes program instructions that, when executed by the associated DP 302A, 304A, 306A, enable the devices to operate in accordance with embodiments of the present disclosure, as detailed herein. It should be appreciated that server 306 can include software stored in its MEM 306B to implement certain aspects of these teachings. In these regards, embodiments of this disclosure may be implemented at least in part by software (or applications) stored on the MEM 302B, 304B, 306B which is executable by DO 302A of UE 302, DP 304A of UE 304, and/or DP 306A of server 306, or by hardware, or by a combination of tangibly stored software and hardware (and tangibly stored firmware). Electronic devices implementing these aspects of the disclosure need not be the entire devices as depicted in FIG. 3, but embodiments may be implemented by one or more components of same such as the above described tangibly stored software, hardware, firmware and DP.

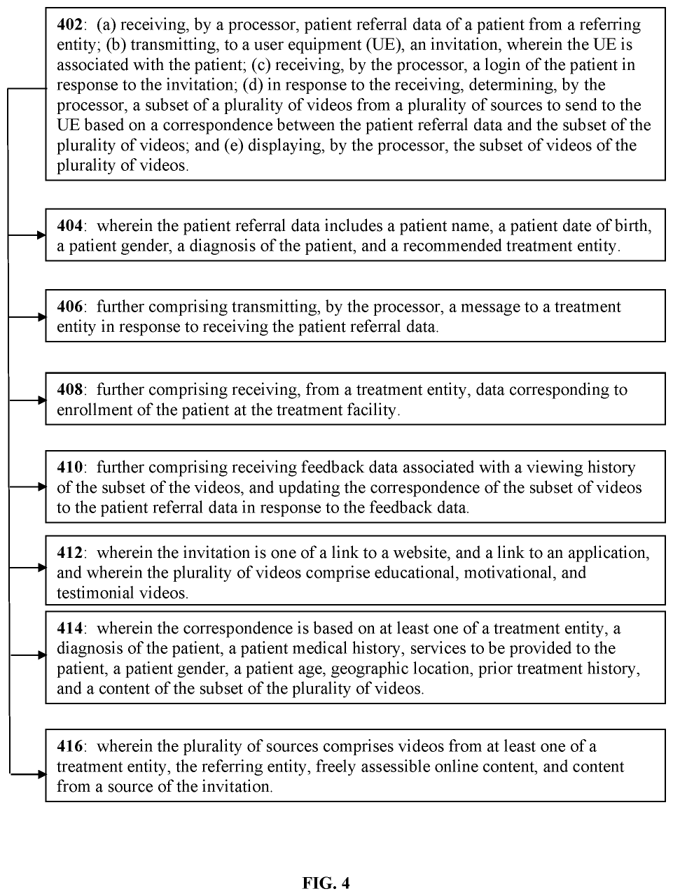

Reference is now made to FIG. 4, which presents a summary of the above teachings for patient referral. Shown in FIG. 4 is an exemplary logic flow diagram in accordance with embodiments of this disclosure. Block 402 presents (a) receiving, by a processor, patient referral data of a patient from a referring entity; (b) transmitting, to a user equipment (UE), an invitation, wherein the UE is associated with the patient; (c) receiving, by the processor, a login of the patient in response to the invitation; (d) in response to the receiving, determining, by the processor, a subset of a plurality of videos from a plurality of sources to send to the UE based on a correspondence between the patient referral data and the subset of the plurality of videos; and (e) displaying, by the processor, the subset of videos of the plurality of videos. Next block 404 states wherein the patient referral data includes a patient name, a patient date of birth, a patient gender, a diagnosis of the patient, and a recommended treatment entity.

Some of the non-limiting implementations detailed above are also summarized in FIG. 4 following block 404. Block 406 indicates further comprising transmitting, by the processor, a message to a treatment entity in response to receiving the patient referral data. Block 408 relates to further comprising receiving, from a treatment entity, data corresponding to enrollment of the patient at the treatment facility. Block 410 states further comprising receiving feedback data associated with a viewing history of the subset of the videos, and updating the correspondence of the subset of videos to the patient referral data in response to the feedback data. Next block 412 indicates wherein the invitation is one of a link to a website, and a link to an application, and wherein the plurality of videos comprise educational, motivational, and testimonial videos. Block 414 then states wherein the correspondence is based on at least one of a treatment entity, a diagnosis of the patient, a patient medical history, services to be provided to the patient, a patient gender, a patient age, geographic location, prior treatment history, and a content of the subset of the plurality of videos. Finally, block 416 recites wherein the plurality of sources comprises videos from at least one of a treatment entity, the referring entity, freely assessable online content, and content from a source of the invitation.

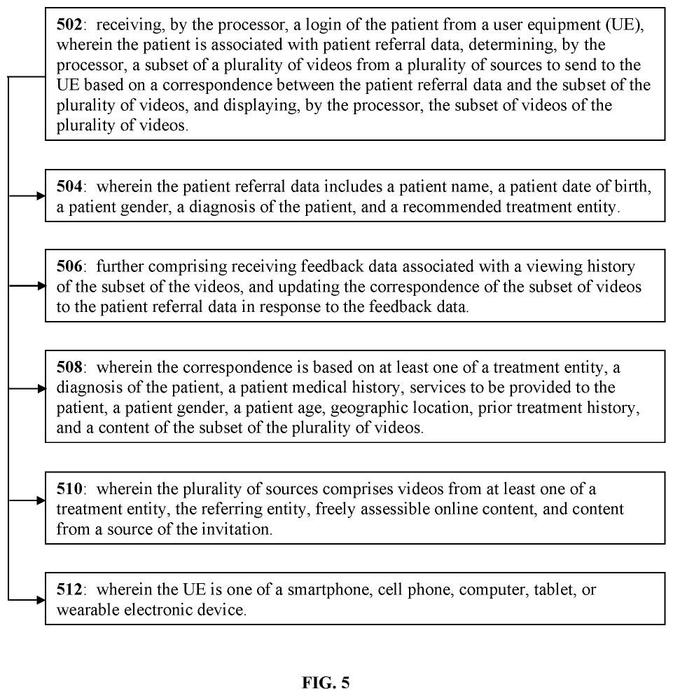

The above teaches are further summarized in the logic flow diagram set forth in FIG. 5 beginning at block 502, which states receiving, by the processor, a login of the patient from a user equipment (UE), wherein the patient is associated with patient referral data, determining, by the processor, a subset of a plurality of videos from a plurality of sources to send to the UE based on a correspondence between the patient referral data and the subset of the plurality of videos, and displaying, by the processor, the subset of videos of the plurality of videos. Block 504 recites wherein the patient referral data includes a patient name, a patient date of birth, a patient gender, a diagnosis of the patient, and a recommended treatment entity.

Next block 506 indicates further comprising receiving feedback data associated with a viewing history of the subset of the videos, and updating the correspondence of the subset of videos to the patient referral data in response to the feedback data. Block 508 states wherein the correspondence is based on at least one of a treatment entity, a diagnosis of the patient, a patient medical history, services to be provided to the patient, a patient gender, a patient age, geographic location, prior treatment history, and a content of the subset of the plurality of videos. Block 510 recites wherein the plurality of sources comprises videos from at least one of a treatment entity, the referring entity, freely assessable online content, and content from a source of the invitation. Lastly, block 512 states wherein the UE is one of a smartphone, cell phone, computer, tablet, or wearable electronic device.

The logic flow diagrams of FIG. 4 and FIG. 5 may be considered to illustrate the operation of a method, or a result of execution of computer program instructions stored in a computer-readable medium. The logic diagram of FIG. 4 and FIG. 5 may also be considered a specific manner in which components of a device are configured to cause that device to operate, whether such a device is a user equipment, server, mobile phone, cell phone, smart phone, laptop, tablet, desktop, wearable electronic device, or other electronic device, or one or more components thereof. The various blocks shown in FIG. 4 and FIG. 5 may also be considered as a plurality of coupled logic circuit elements constructed to carry out the associated function(s), or specific result of strings of computer program instructions or code stored in memory.

Various embodiments of computer-readable medium and computer-readable MEMs 302B, 304B, and 306B include any data storage technology type which is suitable to the local technical environment, including but not limited to semiconductor based memory devices, magnetic memory devices and systems, optical memory devices and systems, fixed memory, removable memory, disc memory, flash memory, dynamic random-access memory (DRAM), static random-access memory (SRAM), electronically erasable programmable read-only memory (EEPROM) and the like. Various embodiments of the processor include but are not limited to general purpose computers, special purpose computers, microprocessors, digital signal processors, and multi-core processors.

Thus, the present disclosure provides for delivering a subset of videos, in ranked or re-ranked order, to a referred patient wherein the videos are directly related to the individual corresponding to a predetermined set of criteria. Specifically, in one configuration, each time the patient opens the present application, the subset of selected videos and order may be adjusted since the prior opening of the application. It is contemplated this re-ordering can be based at least in part upon which videos were reviewed by the patient or a change in the profile and hence ranking of a video based on feedback from other users or if the patient called or made a contact with the treatment facility.

This disclosure has been described in detail with particular reference to a presently preferred embodiment, but it will be understood that variations and modifications can be effected within the spirit and scope of the invention. It is to be understood that any feature described in relation to any one embodiment may be used alone, or in combination with other features described, and may also be used alone, or in combination with one or more features of any other of the embodiments, or any combination of any other of the embodiments. The presently disclosed embodiments are therefore considered in all respects to be illustrative and not restrictive. Furthermore, equivalents and modifications not described above may also be employed without departing from the scope of this disclosure, which is defined in the accompanying claims.

1. A method of providing motivation to get treatment, the method comprising:
(a) receiving, by a processor, patient referral data of a patient from a referring entity;
(b) transmitting, to a user equipment (UE), an invitation, wherein the UE is associated with the patient;
(c) receiving, by the processor, a login of the patient in response to the invitation;
(d) in response to the receiving, determining, by the processor, a subset of a plurality of videos from a plurality of sources to send to the UE based on a correspondence between the patient referral data and the subset of the plurality of videos; and
(e) displaying, by the processor, the subset of videos of the plurality of videos.

2. The method according to claim 1, wherein the patient referral data includes a patient name, a patient date of birth, a patient gender, a diagnosis of the patient, and a recommended treatment entity.

3. The method according to claim 1, further comprising transmitting, by the processor, a message to a treatment entity in response to receiving the patient referral data.

4. The method according to claim 1, further comprising receiving, from a treatment entity, data corresponding to enrollment of the patient at the treatment facility.

5. The method according to claim 1, further comprising receiving feedback data associated with a viewing history of the subset of the videos, and updating the correspondence of the subset of videos to the patient referral data in response to the feedback data.

6. The method according to claim 1, wherein the invitation is one of a link to a website, and a link to an application, and wherein the plurality of videos comprise educational, motivational, and testimonial videos.

7. The method according to claim 1, wherein the correspondence is based on at least one of a treatment entity, a diagnosis of the patient, a patient medical history, services to be provided to the patient, a patient gender, a patient age, geographic location, prior treatment history, and a content of the subset of the plurality of videos.

8. The method according to claim 1, wherein the plurality of sources comprises videos from at least one of a treatment entity, the referring entity, freely assessable online content, and content from a source of the invitation.

9. An apparatus comprising at least one processor and at least one memory storing computer program instructions executable by the at least one processor, wherein the at least one memory and the computer program instructions and the at least one processor are configured to cause the apparatus to at least:
(a) receive patient referral data of a patient from a referring entity;
(b) transmit, to a user equipment (UE), an invitation, wherein the UE is associated with the patient;
(c) receive a login of the patient in response to the invitation; and
(d) in response to the receiving, determine a subset of a plurality of videos from a plurality of sources to send to the UE based on a correspondence between the patient referral data and the subset of the plurality of videos.

10. The apparatus according to claim 9, wherein the patient referral data includes a patient name, a patient date of birth, a patient gender, a diagnosis of the patient, and a recommended treatment entity.

11. The apparatus according to claim 9, the at least one memory and the computer program instructions and the at least one processor are configured to further cause the apparatus to transmit a message to a treatment entity in response to receiving the patient referral data.

12. The apparatus according to claim 9, the at least one memory and the computer program instructions and the at least one processor are configured to further cause the apparatus to receive, from a treatment entity, data corresponding to enrollment (treatment) of the patient at the treatment facility.

13. The apparatus according to claim 9, the at least one memory and the computer program instructions and the at least one processor are configured to further cause the apparatus to receive feedback data associated with a viewing history of the subset of the videos, and updating the correspondence of the subset of videos to the patient referral data in response to the feedback data.

14. The apparatus according to claim 9, wherein the invitation is one of a link to a website, and a link to an application.

15. The apparatus according to claim 9, wherein the correspondence is based on at least one of a treatment entity, a diagnosis of the patient, a patient medical history, services to be provided to the patient, a patient gender, a patient age, geographic location, prior treatment history, and a content of the subset of the plurality of videos.

16. The apparatus according to claim 9, wherein the plurality of sources comprises videos from at least one of a treatment entity, the referring entity, freely assessable online content, and content from a source of the invitation.

17. A non-transitory computer-readable medium tangibly storing computer program instructions which when executed by a processor, cause the processor to at least:
(a) receive patient referral data of a patient from a referring entity;
(b) transmit, to a user equipment (UE), an invitation, wherein the UE is associated with the patient;
(c) receive a login of the patient in response to the invitation; and
(d) in response to the receiving, determine a subset of a plurality of videos from a plurality of sources to send to the UE based on a correspondence between the patient referral data and the subset of the plurality of videos.

18. The non-transitory computer-readable medium according to claim 17, wherein the patient referral data includes a patient name, a patient date of birth, a patient gender, a diagnosis of the patient, and a recommended treatment entity.

19. The non-transitory computer-readable medium according to claim 17, the computer program instructions which when executed by a processor, further cause the processor to at least transmit a message to a treatment entity in response to receiving the patient referral data.

20. The non-transitory computer-readable medium according to claim 17, the computer program instructions which when executed by a processor, further cause the processor to at least receive, from a treatment entity, data corresponding to enrollment (treatment) of the patient at the treatment facility.
